(19) United States
(12) Patent Application Publication
(10) Pub. No.: US 2012/0010584 A1
(43) Pub. Date: Jan. 12, 2012

(54) Absorption Body for Use on Wounds

(75) Inventor: Andreas Schmidt, Duderstadt (DE)

(73) Assignee: MCAIRLAID'S VLIESSTOFFE GMBH & CO. KG, Berlingerode (DE)

(21) Appl. No.: 13/178,580

(22) Filed: Jul. 8, 2011

(30) Foreign Application Priority Data
Jul. 9, 2010 (DE) 20 2010 008 025.8

(51) Int. Cl. A61F 13/511; A61F 13/514; A61L 15/22

(52) U.S. Cl. 604/372; 604/358; 604/383

(57) ABSTRACT

An absorption body for use on wounds is constructed of a multilayer configuration with a first layer that is facing the wound, a second layer that is facing away from the wound, and an absorbent core that is arranged between the first and second layers. The first layer in the area of the wound has a material that is permeable for liquids and the second layer has a breathable material. The absorption body is suitable as a wound pad.

### BACKGROUND OF THE INVENTION

The invention concerns an absorption body to be placed onto a wound and comprised of a layered body of a multi-layer construction, comprising a layer A that is facing the skin, a layer B that is facing away from the skin, and an absorbent body or core that is arranged between these layers A and B.

Absorption bodies for absorbing bodily liquids are not only used in hygiene but also in medicine, in particular for treatment of wounds.

Absorption bodies that are used as wound pads are generally placed onto an external wound in order to prevent penetration of foreign bodies into the wound and to absorb blood and ichor.

U.S. Pat. No. 7,335,809 B2 discloses an absorption body for placement on wounds, in particular for absorbing ichor. The illustrated absorption body comprises a flat material section of absorption material, i.e., an absorbent nonwoven with superabsorbent particles distributed therein, as well as a liquid-permeable envelope. This liquid-permeable envelope surrounds the material section of absorption material and forms a barrier against solid body secretions and prevents passage of other excreted substances into the absorbent material arranged within the envelope. The material section of absorption material has a surface that is smaller than the surface of the envelope, i.e., in the unwetted state 3% to 75% of the laid-flat envelope. The surface of the laid-flat envelope is moreover delimited by a seam and optionally by a folded edge. The envelope and/or the absorption material can be provided at its/their circumference with a substance that adheres to the body of the patient, such as an adhesive. As a suitable adhesive, pectin-cellulose compounds are mentioned.

The wound pads disclosed in the prior art have the disadvantage that they must be secured on the wound by means of an appropriate bandage. They have no suitable means with which the wound pad can be secured directly on the skin of the patient.

The present invention has therefore the object to provide an absorption body for placement onto wounds that has full absorption capacity but can be fixed also without further auxiliary means such as bandage material on the wound or on the skin of the patient.

### SUMMARY OF THE INVENTION

Object of the present invention is an absorption body of the aforementioned kind that is characterized in that the layer A in the area of the wound has a material that is permeable for liquids and the layer B comprises breathable material.

The absorption body according to the invention is suitable in particular for placement onto wounds, i.e., onto so-called external wounds which generally may occur on the skin, including the mucous membrane, of the patient. These wounds can be acute injuries but also chronic wounds, i.e., wounds that do not heal or heal only by secondary wound healing, from which e.g. ichor exits.

A further object of the present invention is therefore the use of the absorption body as a wound pad.

The absorption body according to the invention has the advantage that the wound is covered by a layer that takes up the exuded bodily liquid, which layer takes up the exuded liquid or through which the liquid passes and is transported into the neighboring absorbent body or core in which the liquid is absorbed. The layer that is facing away from the skin has the advantage that it is breathable, i.e., permeable to water vapor but impermeable to larger molecules or particles. Perspiration can escape from the interior to the exterior of the absorption body placed on a wound. The wound that is covered by the absorption body according to the invention is not hermetically sealed; air and moisture exchange can take place, ichor and other liquids however cannot penetrate to the exterior and can also not pass from the exterior in the direction toward the wound. This has also the advantage that the breathable layer constitutes a barrier also for bacteria etc. which could infect the wound.

The absorption body according to the invention is a layered body of a multi-layer construction that is usually comprised substantially of a layer A that is facing the skin, a layer B that is facing away from the skin, and an absorbent body that is arranged between these two layers.

The layer A that is facing the skin is positioned immediately on the wound and on the skin surface surrounding the wound. In order to ensure an optimal wound care, the layer A, at least in the area in which it is resting immediately on the wound, is comprised of a liquid-permeable material. This liquid-permeable material can be selected from materials such as nonwoven or woven and/or perforated two-dimensional or three-dimensional films that preferably do not stick to wounds. Nonwoven materials are textile-like materials that are produced from long fibers and are connected to each other by means of chemical, mechanical, thermal or solvent treatment. They are thus textile flat structures of individual fibers and are liquid-permeable. In addition to the nonwovens (=fleece materials), also perforated or structured two-dimensional or three-dimensional films can be used as a layer A. As a two-dimensional configuration, the perforated films have holes in the surface that ensure that liquid can pass through the material. Perforated three-dimensional films are characterized in that by the perforations the perforated material extends across the plane of the flat structure so that a three-dimensional structure is produced. The three-dimensional structure reduces the contact surface area on the wound; this may further positively affect wound healing and prevents sticking of dried exudate. The materials from which the nonwoven materials or the perforated films are produced are preferably polyolefin films, such as polyethylene or polypropylene films or also nonwovens of natural material.

In a possible embodiment of the present invention, the layer A in the area outside of the wound can be provided at least partially with an adhesive. In this way, the absorption body can be secured or fixed on the body surface.

As adhesives all conceivable skin-compatible adhesives are conceivable as they are conventional and known in the production of adhesive plasters. Preferably, the adhesive should be a permanently adhesive material that even after a single use, i.e., when already applied once onto the skin surface and removed, can still be reused. Also, the adhesive, after contacting water, should preferably not lose its adhesive power. A preferably employed adhesive is so-called underindexed polyurethane, i.e., a polyurethane with low isocyanate index. Underindexed polyurethanes are obtained by reaction of polyisocyanates with long-chain polyols that are preferably free of any short-chain proportions.

The topside of the absorption body according to the invention, i.e., the side that is facing away from the skin is formed by layer B. Between the layers A and B the absorbent body or core is arranged. The layers A and B as well as the absorbent body can have the same dimensions. In a possible embodiment the dimensions of the absorbent body are smaller than that of the layers A and B. In this embodiment the absorbent body is essentially enclosed by the layers A and B. Preferably, the layers A and B have the same dimensions and the absorbent body is smaller than these two layers A and B so that the layers A and B project past the absorbent body. At these projecting portions the layers A and B can be connected to each other. In order to connect individual layers to each other, they can be adhesively connected, welded, sealed or connected in other ways, depending on the material from which these layers are formed. In a possible embodiment the dimensions of one of the two layers A or B is greater than that of the other layer, the projecting edges can therefore be folded over about the edge of the smaller layer so that the edges can also be dosed.

An optimal wound care is possible in particular when the layer that is facing away from the skin is a layer of breathable materials. The materials from which the layers A and B are produced, can be the same or different from each other. According to the invention, the layer B is breathable, i.e., it is permeable for water vapor (perspiration) but not for liquids. The layer B can be, for example, a breathable film such as a perforated two-dimensional and/or three-dimensional film, a breathable SMS (spunbond/meltblown/spunbond), nonwoven (fleeces) of natural or synthetic fibers, and/or a laminate of different materials, such as nonwoven and breathable film (BTBS films=breathable film textile backsheet), as they are conventionally used for producing external sides of incontinence products.

As a third layer, the absorption body according to the invention has an absorbent core. It is arranged between the layers A and B, preferably enclosed in the manner of an envelope by the layers A and B. The absorbent core serves for absorbing the exudates that are exiting from the wound and to absorb them permanently. A re-wetting in the direction of the wound should be prevented as much as possible. The absorbent core is therefore preferably formed of a material that is capable not only of absorbing bodily liquids but also of storing them, such as pulp or pulp-related materials as well as synthetic absorbent materials. The absorbent core is preferably a nonwoven of pulp fibers such as an airlaid.

In a preferred embodiment an absorbent core is used that is produced in accordance with the method described in European patent 1 032 342. Preferably, an airlaid of pulp fibers is used as absorbent material. In a preferred embodiment of the present invention, the absorbent core has, distributed across its surface, embossed areas in which the fibers, preferably pulp fibers, are more strongly compressed with each other than in other areas and, in this way, are connected to each other without adhesive and/or binding agent. In such an embodiment, the absorbent body is produced of a fiber material web of pulp fibers which, with formation of an embossed pattern in the compressed areas, are calendered and connected without binding agent in a spot-shaped or line-shaped pattern.

The absorbent core can have the embossed areas (be provided with the embossed areas) on the surface that is facing the layer A and/or the layer B. The fiber layer of the absorbent core is thus structured such that the pulp fibers outside of these discrete embossed areas are present in loosely placed form or only weakly adhering to each other, while in the embossed areas they are compressed with each other and are in intimate connection with the neighboring pulp fibers. With this embodiment, the use of adhesives and binding agents is completely unnecessary for formation of the composite of pulp fibers; this enables simple and complete recycling. In the embossed areas the fibers do not simply adhere to each other. Instead, because of pressure loading in the embossed areas, it is achieved that neighboring pulp fibers in these embossed areas are fixedly connected to each other. This connection is capable of resisting the action of moisture so that the absorption body according to the invention is distinguished by mechanical load capacity even in the wet state.

The areas outside of the discrete embossed areas, in which embossed areas the fibers are present in loosely laid form on top of each other or in a form where they only weakly adhere to each other, are characterized by an excellent absorption capacity. The absorbed liquid is absorbed by the fibers and is distributed across the entire surface area of the absorbent core and held therein.

In order to further increase the absorption capacity of the absorbent core, the absorbent core may contain particles of superabsorbent polymers (SAPs). They can be, for example, directly worked in during manufacture of the absorbent core.

The absorbent body C is arranged between the layers A and B and preferably enveloped by the layers A and B in the manner of an envelope. The absorbent body can be secured on one or both of the layers A and B or can be loosely enveloped by layers A and B. In order to avoid as much as possible sliding of the absorbent body within this envelope and to ensure that the absorbent body at least completely or partially covers the layer A at the location where it is resting on the wound, the absorbent body should have at least the same size as the wound. In a preferred embodiment, the absorbent body is smaller than the layer A.

The layers A and B are preferably closed at their outer edges. In a possible embodiment of the present invention the layers A and B are identical or almost identical and the edges are adhesively connected, welded or sealed to each other. This connection of the two layers can be realized immediately at the edges of the layers but also at a certain spacing away from the edges so that essentially a circumferentially extending seam is formed. An adhesive connection extending up to the adhesive core is also conceivable.

In a further possible embodiment of the present invention, the surface of one of the two layers A and B is greater than the other; the projecting surfaces of the larger layer can be folded about the edges of the smaller layer so that an envelope is produced also that encloses the absorbent body.

In order to increase the stability of the absorption body according to the invention, in a preferred embodiment of the present invention the absorbent body of the layer C and the layer B that is facing away from the skin are adhesively connected to each other, i.e., preferably glued. Gluing of the layers B and C prevents sliding of the absorbent core or body within the absorption body or wound pad and also provides stability of the absorption body (e.g. wound pad) as such. A stable and resilient product is obtained that usually will not burst even under load. Moreover, the adhesive connection also has advantages with respect to manufacturing technology; while a circumferentially extending adhesive edge allows only for minimal manufacturing tolerances, the manufacturing tolerances are significantly greater for partial or complete surficial adhesive connection.

### BRIEF DESCRIPTION OF THE DRAWINGS

The present invention will be explained in more detail with the aid of the attached figures.

FIG. 1 shows a plan view onto the absorption body.

FIG. 2 shows an embodiment of the absorption body in a section illustration.

FIG. 3 shows another embodiment.

FIG. 4 shows yet another embodiment.

FIG. 5 is a detail view of a section through the absorbent core.

FIG. 6 is a perspective illustration of a section of an absorbent core.

### DESCRIPTION OF PREFERRED EMBODIMENTS

FIG. 1 shows the absorption body 1 according to the invention. The layer 2 (layer A) facing the skin/the wound and a circumferentially extending outer area in which the layer 2 that is facing the skin and the layer 3 (layer B) that is facing away from the skin are connected to each other are illustrated. The layer 2 that is facing the skin is provided on the outer area 4 with an adhesive. The adhesive should be applied only to those areas that will not come into contact with the wound itself. In order not to impair the absorption capability of the absorption body according to the invention, the surfaces of the layer 2 facing the skin should be as much as possible free of any coatings etc. so that the liquid permeability of this layer is maintained.

In FIG. 2 a possible embodiment of the absorption body according to the invention is illustrated in a schematic section view. The absorption body 1 is a layered configuration of a multi-layer construction with a layer 2 facing the skin and a layer 3 that is facing away from the skin, the layers being connected to each other along their edge areas 4. The wound contact surface 5 is liquid-permeable in order to allow passage of exudate that is being excreted from the wound. These exudates are absorbed by the absorbent body 6 and stored therein. As a further layer, the absorption body has on the side that is facing away from the skin the layer 3 that forms the cover layer of the absorption body 1. The cover layer 3 is formed of breathable materials, i.e., those materials that are permeable for vapor but are liquid-impermeable. By means of this cover layer facing away from the skin it should be prevented as much as possible that exudate will escape from the absorption body and will soil clothing, for example. In the embodiment illustrated here, the absorbent body 6 and the layer 3 that is facing away from the skin are glued to each other; the adhesive is illustrated here as an adhesive layer 9.

In the embodiment illustrated in FIG. 2, the layer 2 that is facing the skin and the layer 3 that is facing away from the skin are connected to each other in the area 4 and form a circumferentially extending edge. The connection of the layers can be spot-shaped so that the layers 2 and 3 are not completely connected to each other in the outer areas 4. As illustrated here, they can also be connected to each other across the entire surface area. In the area 5 the absorption body is resting on the skin H; this area extends across the entire wound and preferably also across the area of the skin that is immediately adjacent to the wound and possibly also beyond. In the embodiment illustrated in FIG. 2, the absorbent body 6 is glued onto the layer 3 that is facing away from the skin.

In the embodiment illustrated in FIG. 3, the absorbent body extends up to the outer edges 4 where, for connection of the layers 2 and 3, it is also engaged and therefore secured between these layers.

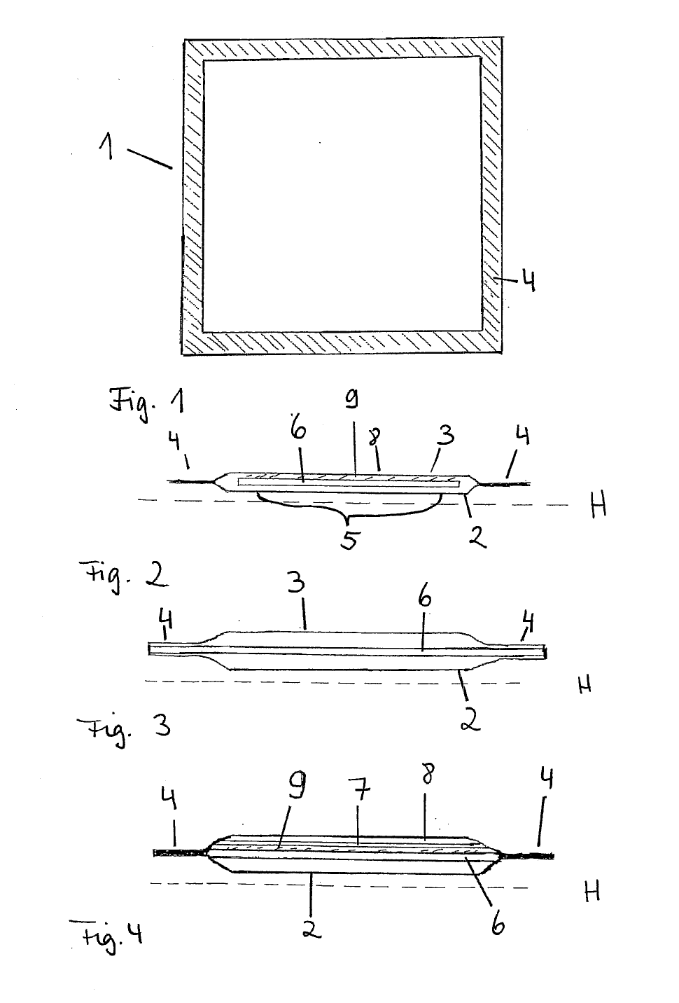

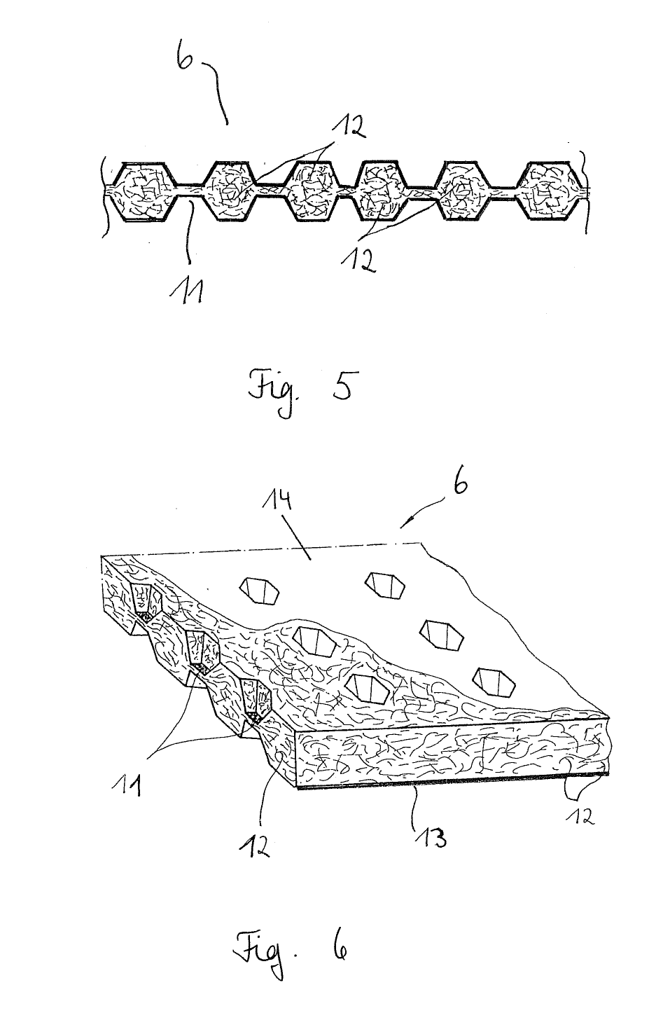

In FIG. 4, a further embodiment of the absorption body according to the invention is illustrated. In this embodiment, the layer 3 that is facing away from the wound/skin is comprised of a laminate. In this connection, the breathable layer 7 is facing the absorbent body; the termination to the exterior is formed by an additional layer material in the form of a nonwoven 8. In a possible embodiment, the outwardly oriented side of the breathable layer 7 can be printed with a pattern, logo, or the like which, in the finished laminate, will then shine through to the exterior. The layers 7 and 8 can also be connected to each other adhesively, for example, can be glued to each other. In the embodiment illustrated here, the layer 3 that is facing away from the skin and the absorbent body 6 can be glued to each other, here illustrated by adhesive layer 9.

In FIG. 5, in a detail illustration a possible embodiment of the absorbent body 6 in cross-section and, in FIG. 6, a perspective illustration of a section of the absorbent body 6 are illustrated.

The layer 10 that determines the absorption behavior for liquids, in particular ichor, is comprised preferably of pulp fibers 11. The pulp fibers 11 are compressed in the embossed areas 12 and are connected to each other in this way. In the embodiment illustrated here, the embossed areas 12 are located opposite each other on the top side and bottom side so that in the respective embossed area 12 only a narrow stay of interconnected pulp material remains. The other areas of the layer 10 arranged between these embossed areas 12 have a loosely laid pulp layer. An intimate connection between the pulp fibers 11 does not exist in these areas.

The embossed areas 12 have in the embodiment illustrated here the shape of truncated pyramids or truncated cones, wherein the angle of the thus formed slanted sides should be between 10° and 45°.

As cellulose material for the layer 10 inexpensively available mass-produced material can be used. Preferably, if possible, unbleached so-called “fluff pulp” is used that, in contrast to bleached pulp materials, is characterized by an excellent binding behavior; this improves mechanical strength of the absorbent body with regard to vertical tensile forces.

When manufactured in a continuous method, the fiber material web that later on forms the layer 10 is manufactured of a layered arrangement of pulp fibers of defiberized pulp (wood pulp) loosely laid in an air stream and optionally interspersed absorbent materials. The fiber material web of pulp fibers is obtained by layered laying of the pulp fibers. When further absorbent materials, such as SAP (superabsorbent polymers), are added, this material addition can be done also by layered laying, for example, by alternatingly laying pulp fibers, absorbent material, pulp fibers until the desired quantity of pulp fibers and optionally absorbent material has been supplied. In the embodiment of an absorbent body, illustrated in FIG. 6, the layer 10 of pulp fibers 11 has on its top side and/or bottom side a further layer 13, 14, respectively. These layers 13, 14 are preferably made of a thin tissue material. This material is already incorporated upon manufacture of the absorbent core employed according to the present invention in that the pulp fiber layer is applied to the tissue 13 and covered by a further tissue 14. The layered arrangement of tissue and pulp as well as optionally a further tissue layer can be introduced together into a calandar with which a pattern of spot-shaped or line-shaped pressure areas is generated. This method is disclosed, for example, in European patent 1 032 342.

The tissue material is known in the prior art and can be obtained commercially as web material. The tissue material imparts additional stability to the absorbent body 6; it can also prevent that fine wear debris of the pulp fibers and/or of the absorbent materials can penetrate in the direction toward the wound through the layer A that is permeable for liquids.

For manufacturing a standardized defiberized product, it is possible to use commercially available renewable wood resources.

The method for layering pulp as a starting material for the layer 10 enables dry processing of the pulp fibers 11 and thus an excellent fusion of the pulp fibers in the discrete embossed areas by means of the subsequent embossment process between two structured rollers. Outside of the embossed areas 12 the fibers are loosely resting on each other; this improves the absorption capacity and flexibility of the layer 10.

The absorption behavior of the layer 10 is substantially “dry”, i.e. as long as a complete saturation has not been reached, the absorbed liquid cannot be squeezed out as it is possible with a sponge. Even after use and as long as the absorption behavior of the absorbent body 6 has not been exhausted, the absorption body therefore appears dry.

Manufacture is done on the basis of web material which is produced in a continuous process. In air-supported laying (air laying), first the pulp fibers and the SAPs for forming the layer 10 are laid. Subsequently, in a calandar with two structured calandar rollers the production of the embossed areas 12 is realized. In further continuous steps the top layer 3 above and the bottom layer 2 below are arranged and adhesively connected with the core layer 10. The thus produced web material is subsequently cut into individual, preferably rectangular, fields that are supplied in a further manufacturing step for producing the absorption body according to the invention.

The specification incorporates by reference the entire disclosure of German priority document 20 2010 008 025.8 having a filing date of Jul. 9, 2010.

While specific embodiments of the invention have been shown and described in detail to illustrate the inventive principles, it will be understood that the invention may be embodied otherwise without departing from such principles.

What is claimed is:

1. An absorption body for use on a wound, the absorption body comprising a layered body of a multi-layer configuration, comprising
a first layer that is to face a wound;
a second layer facing away from the wound;
an absorbent core arranged between the first and second layers;
wherein the first layer in the area of the wound comprises a first material that is permeable for liquids and the second layer comprises a second material that is breathable.

2. The absorption body according to claim 1, wherein the first layer is liquid-permeable and is comprised of at least one material selected from nonwoven, woven, a perforated two-dimensional film, and perforated three-dimensional film.

3. The absorption body according to claim 1, wherein the absorbent core comprises a nonwoven of a liquid-storing pulp or a liquid-storing pulp-related material.

4. The absorption body according to claim 3, wherein the absorbent core contains particles of superabsorbent polymers.

5. The absorption body according to claim 1, wherein the breathable material of the second layer is selected from the group consisting of films, nonwovens, and laminates.

6. The absorption body according to claim 5, wherein the films are perforated films or breathable SMS films.

7. The absorption body according to claim 5, wherein the nonwovens are comprised of natural fibers or synthetic fibers.

8. The absorption body according to claim 5, wherein the laminates are comprised of a nonwoven and a breathable film and/or a BTBS film.

9. The absorption body according to claim 1, wherein the first layer and second layer envelope the absorbent core and wherein the first and second layers have edges that are closed.

10. The absorption body according to claim 1, wherein the absorbent core is a flat structure that has dimensions that are smaller than dimensions of a surface of the first layer or the second layer.

11. The absorption body according to claim 1, wherein the absorbent core is comprised of pulp fibers provided with embossed areas distributed across a surface area of the absorbent core, wherein in the embossed areas the pulp fibers are more strongly compressed with each other than in remaining areas that remain between the embossed areas, wherein the pulp fibers are connected without an adhesive and/or a binding agent in the embossed areas.

12. The absorption body according to claim 11, wherein the embossed areas are of a truncated cone shape or a truncated pyramid shape.

13. The absorption body according to claim 1, wherein the absorbent core comprises an absorbent layer of fibers of defiberized pulp that are layered in an air stream and further comprises a first tissue layer and a second tissue layer arranged above and below the absorbent layer, respectively.

14. The absorption body according to claim 1, wherein the first material on a surface of the first layer that is facing the wound comprises a nonwoven, a woven, a perforated two-dimensional film, or a perforated three-dimensional film, each configured not to impair breathability.

15. The absorption body according to claim 1, wherein the second layer comprises an additional layer material that is facing away from the wound and wherein the breathable material of the second layer and the additional layer material are adhesively connected to each other.

16. The absorption body according to claim 1, wherein the second layer and the absorbent core are adhesively connected to each other.

17. The absorption body according to claim 1 as a wound pad.
